(19) United States
(12) Patent Application Publication
(10) Pub. No.: US 2003/0125745 A1
(43) Pub. Date: Jul. 3, 2003

(54) Bone-fixing device

(75) Inventors: How Tseng, Taipei (TW); Jiunn-Liang Chen, Taipei (TW); Chao-Yu Chen, Taipei (TW)

Correspondence Address: David E. Dougherty, 612 Crystal Square 4, 1745 Jefferson Davis Highway, Arlington, VA, 22202-3417, US

(73) Assignee: Bio One Tech Inc.

(21) Appl. No.: 10/265,609

(22) Filed: Oct. 8, 2002

(30) Foreign Application Priority Data
Nov. 5, 2001 (TW) 90127389

(51) Int. Cl. A61B 17/56

(52) U.S. Cl. 606/73

(57) ABSTRACT

A bone fixing device includes a screw and a screwdriver each of which is made of non-magnetic and bioabsorable materials. The screw has a head formed with at least one slot and a threaded portion separated from the head by a neck. The screwdriver cooperates with the slot to apply a force to the screw.

The present invention is related to a bone-fixing device, especially a biocompatible screw and a complementary screwdriver; the screw comprises a shank, a neck and a head with at least one straight slot; the screw is made of non-magnetic bioabsorbable material and cooperates with the screwdriver for achieving the intended effects of clamping and central fixation.

### FIELD OF THE INVENTION

The present invention is related to a bone-fixing device, particularly to a biocompatible screw and a complementary screwdriver.

### BACKGROUND OF THE INVENTION

At present, metal bone plates or nails are mostly used for fixation (Muller et al., 1979; Schaztker & Tile, 1987) in treatment of bone fracture. However, the use of metal devices consists of the following disadvantages:

(1) Corrosion may occur to the implant after a period of time causing release of ions or particles into the surrounding tissue, thereby causing inflammation, infection or other complications. A secondary surgery may be needed to remove the implanted device.

(2) The stiffness of the metal device per se prevents the periosteal callus from forming and causes delayed union or nonunion.

(3) The stiffness of the metal device per se being much higher than human bones (where human bone is ca. 120 MPa, titanium is ca. 1250 MPa, stainless steel is ca. 850 MPa, and cobalt chrome alloy is ca.700 MPa) will result in stress shielding (Tonino et al., 1976) causing the bones to lose normal pressure stimulus, such that under extensive stress protection, bone cell depauperation will occur (Cochran, 1969; Tonino et al., 1976; Uhthoff & Dubuc 1971, Slatis et al., 1978) thereby further producing osteoporosis. The mechanical property of bones may, thus, deteriorate and consequent fracture is very likely to occur when the metal device is removed.

Ceramic with excellent stability may be implemented to solve the problem of metal corrosion (Kawahara et al., 1980), but the Young's moduli of ceramic being as high as 400 GPa, much higher than that of the metal, averting ceramic or metal material from being a suitable biocompatible material for bone fixation.

Investigation has been made in this field to find out that the ideal bone-fixing materials must have the following characteristics:

(1) Excellent biocompatibility which does not result in allergic, immune, or cancerous responses locally or systematically allowing bones to reset successfully.

(2) Similar elasticity to bones allowing formation of the periosteal callus without delayed union or nonunion.

(3) Sufficient mechanical strength to avoid break or failure at the initial stage, while the material is bioabsorbable or bioresorbable to ensure the stress shift gradually to bones in union process without needing a secondary surgery to remove the implant.

Such a bioabsorbable material has become a direction in the research of bone materials.

Information shows that development of bioabsorbable bone-fixing device starts with jaw and facial surgery in which marco-molecular materials have been adopted. Poly (alpha-hydroxy acid) is one of the most remarkable materials since 70s in this field which has satisfied the requirements to serve as an ideal bone-fixing material, due to its good biocompatibility, proper stiffness, its characteristics of leaving no residue of small particles in the body after decomposition and being absorbable (Higgins, 1954; Leenslag, 1982). Various configurations and shapes have been prepared for repairing hard structures of living bodies.

In the history of hard structure repair, screws provided great convenience but involved the following problems in terms of biological applications:

(1) Most metal screws are not suitable for medical applications:

To enhance the performance of screws, most techniques place emphasis on different sizes and shapes of metal screws. However, screws are mostly featured with high specific weight, high price and easy corrosion. They are not accepted, not to say absorbed, by the bio bodies except for very few titanium and special alloy screws, such that screws can hardly be used in the medical field.

(2) Hard to fix

The existing techniques used in bone repair and regeneration include absorbable screws each having a shank, a head and a slot formed on the head for mating with a screwdriver. Though absorbable by living bodies, the engagement between a screw and a screwdriver is not good enough due to their poor designs. For example, a surgeon during the surgery is not supposed to need an additional hand or other tools to complete the installation once a screwdriver has picked up a screw. However, the screw often drops because of the poor engagement between the screw and the screwdriver. As the result, the surgery is far from smooth running, or rather in higher risk of infections.

(3) Easy fracture of screw head

Despite of not being as strong as metal screws in strength and shear strength, bio-absorbable screws have important functions in medical applications, the most important one of which is that they can be absorbed and accepted by living bodies. The strength must be designed up to the maximum in order to counteract the relative weakness of bioabsorbable materials as compared with metals. Screw heads designed in the past fractured easily in the process of being driven into bones due to the excessive torque or strength applied to materials.

(4) Insufficient locking strength

The locking torque is concentrated on the screw threads when a screw is implanted into the bone plate and the bone, and the screw often become loose due to insufficient locking strength. To overcome such a problem, more screws are used for reinforcement in many cases in the past.

(5) Screws in complicated varieties

The most common types of screws (such as minus-type, Phillips type, inner hexagonal, and quincunx) and other special types of screws must match with their corresponding screwdrivers. The more complicated the profiles of the screw heads, the more difficulty and higher cost must be involved in make the screws and screw drivers.

### SUMMARY OF THE INVENTION

The primary object of the present invention is to provide a Bone-fixing Device mainly comprising a screw and screwdriver each made of non-magnetic and bioabsorbable materials, wherein said screw has a head formed with at least one slot and a threaded neck separated from the screw head by a neck; said screwdriver cooperates with the slot for apply a force to the screw.

The object of the present invention is to provide a bone-fixing device with a high intensity screw. It has been wished in the past that a minimum force is exercised to obtain a maximum torque. Failure of head easily happens under the maximum torque due to the limits of the material per se, which failure may often found at the head or even the threads. This invention provides a neck between the head and the shank that joins to the head at its top end and to the shank at its bottom end. The neck evenly distributes the force being applied so as to maximize the torque, which the screw may sustain to avoid break of the joint when the damage is very likely to take place under the original force being applied. Every joint of slant facet is rounded for better appearance and for reducing stress concentration. The designs of the screw neck and the rounded facets greatly increase the resistance to damages caused by high torque.

The object of this invention is to provide a bone-fixing device with consolidated screws. The screw neck refers to the joint between the head and the shank. To avoid disengagement of screw under the stress concentrating on the shank when implanted, dimensions of the neck and the pilot hole on bone plate are set to be identical for producing friction between the neck and the hole on the bone plate, in addition to the locking force provided by the shank so as to allow the screw to be affixed to the bone plate and the bone for a longer period of time.

The object of the present invention is to provide a bone-fixing device with tight engagement between said screw and screwdriver. With such tight engagement, loose of screw is likely to happen due to human errors leading to more difficult surgery and more risk of infections. In the present invention, surface friction obtained by the tight engagement of the screw slot and the slant facet makes it much easier for handy manipulation. The successful rate is higher with the relatively short time that patients remain on the surgical table.

The object of the present invention is to provide a bone-fixing device that can be completely embedded into the bone plates. In the past, it was quite often that the head could not be entirely embedded in the bone plates during the engaging process or, the socket driver could not make a full embedding in the bone plates due to wedges between the socket configuration and the pilot hole. The bulges result in unpleasant appearances for patients after the surgery. Perfect matching of bone plates with the screw heads is provided in this invention for a smooth implant allowing the screw to be completely embedded into the bone plates.

The object of this invention is to provide a bone-fixing device capable of center positioning. A positioning rib having a depth and width equal to those of the slot is located at the center of the screw slot for properly guiding the screwdriver to the center position without going sideways, so as to obtain a maximum torque by applying a minimum force. The vertical structure under the rib increases contact area with the screwdriver so as to attain tight engagement both in X and Y directions. The force applied by the screwdriver is properly delivered to the screw without producing any unnecessary force component.

### BRIEF DESCRIPTION OF THE DRAWINGS

The following drawings are attached for illustration of several embodiments with the wish to further introduce its structure, features, functions and objects of this invention.

FIG. 1 is a partial structure of a minus-type screw and screwdriver in accordance with the first embodiment of the present invention;

FIG. 2 is an overall structure of a minus-type screw and screwdriver in accordance with the first embodiment of the present invention;

FIG. 3 is a partial structure of a Phillips-type screw and screwdriver in accordance with the first embodiment of the present invention;

FIG. 4 is a cross-sectional view of a minus-type screw in accordance with the first embodiment of the present invention;

FIG. 5 is a cross-sectional view illustrating a minus-type screw and screwdriver prior to joint in accordance with the first embodiment of the present invention;

FIG. 6 is a cross-sectional view illustrating a minus-type screw and screwdriver after engagement in accordance with the first embodiment of the present invention;

FIG. 7 is illustrates a minus-type screw in the process of being locked into bone plates and bones of the first embodiment of the present invention;

FIG. 8 is illustrates a minus-type screw after being locked into bone plates and bones of the first embodiment of the present invention;

FIG. 9 is a cross-sectional view taken along Lines 9-9 in FIG. 8 illustrating the first embodiment;

FIG. 10 is a cross-sectional view showing a traditional minus-type screw after being locked into a bone plate;

FIG. 11 is a cross-sectional view showing a minus-type screw of the present invention after being locked into a bone plate;

FIG. 12 is a schematic view showing the positioning rib provided in the screw slot in accordance with the second embodiment of the present invention;

FIG. 13 is a cross-sectional view showing various positioning ribs in accordance with the second embodiment of the present invention;

FIG. 14 is a perspective view showing a screw and a screwdriver prior to engagement in accordance with the third embodiment of the present invention; and

FIG. 15 is a perspective drawing showing a screw and a screwdriver after engagement in accordance with the third embodiment of the present invention.

### DETAILED DESCRIPTION OF PREFERRED EMBODIMENTS

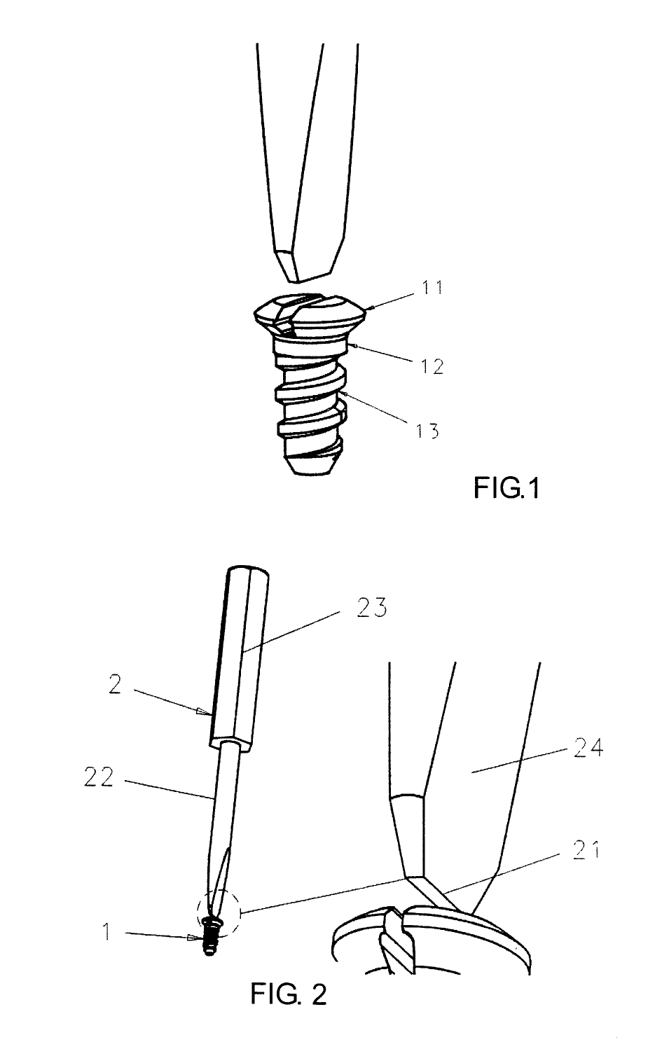

Referring to FIGS. 1 and 2, the present invention comprises a biocompatible screw 1 and a matching screwdriver 2, wherein the screw 1 comprises a head 11, a neck 12, and a shank 13. The head 11 is formed with a slot 3 running along its width, forming a minus-type screw of the first embodiment of the present invention; the screwdriver 2 has a tip 21, a shank 22 and a handle 23, as shown in FIG. 2; the tip 21 is to be inserted into the slot 3 of the minus-type screw for applying a torque to the screw 1.

The number of slots 3 is subject to configuration with other functions and not limited to the function in the first embodiment. The number of slot in this invention should not exceed three to avoid over torque and failure of the head 11 or even the threads, in consideration of the material used herein being softer than metal. The number of slots can be increased to six or eight if the design requires or a different material is used in this invention.

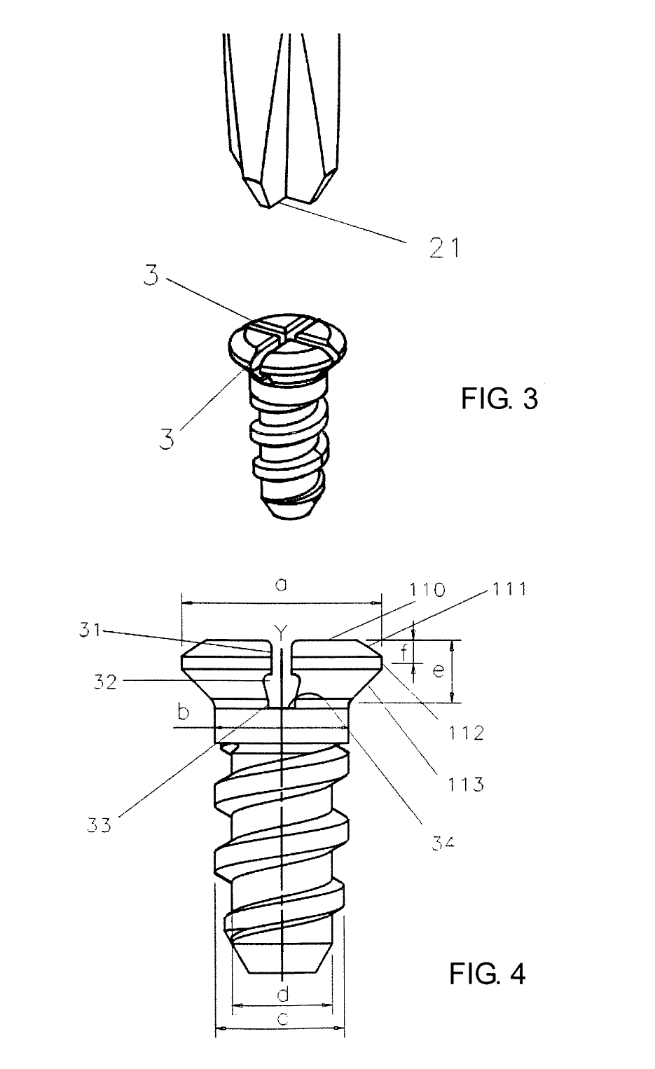

With reference to FIG. 3, if the head has two slots normal and symmetrical to each other, the screw is the so-called Philips-type as shown in the first embodiment. Regardless of the number of slots 3 on the head11, said screwdriver 2 should match with the number and shape of the slots. For example, a Philips-type tip 21 is provided to match with the Phillips-type screwdriver 2, for matching the Philips-type slots of the Philips-type head 11.

FIG. 4 shows a minus-type screw of the first embodiment, wherein the head 11 and the shank 13 is separated from each other by a neck 12. The head includes a flat top 110, an upper ring 111, a middle ring 112 and tapered lower ring 113, the screw neck 12 is basically a cone structure, extending downwards from the tapered lower ring 113 to the shank 13 to form an integral body. Diameter “a” of the head, diameter “b” of the neck, major diameter of and minor diameter of the shank can be obtained by measurements taken along the cross-section of the central axis Y.

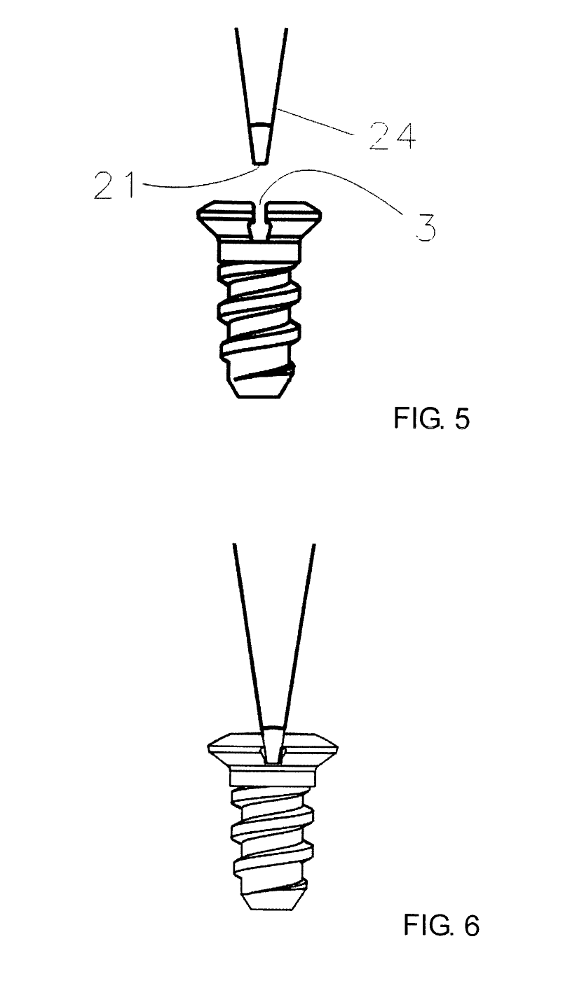

The slot 3 of the head 11 comprises of a holding section 31, a middle section 32 and a fixing section 33 from top to bottom. Referring to FIGS. 5 and 6, when the tip 21 of the screwdriver 2 is inserted downward to the slot 3, it first touches the holding section 31 when the said cone slant 24 of the tip 21 is inserted downwards into the holding section 31, the holding section 31 is enlarged by the slant 24 because the screw 1 per se is made of macro molecular material, which has recovering elasticity. As a result, the screw 1 clamps tightly to said tip 21 of side screwdriver 2. Upon passage of the tip 21 through the middle section 32 of the slot 3, the screw 1 deforms to release stress; and again upon passage of the tip 21 down into the fixing section 33 of the slot 3, the screw is finally positioned at the bottom without any swing. Thus, when used during surgeries, the screw is picked up by the screwdriver without using the other hand or other aid for installation; otherwise the poor engagement between the screw and the screw driver can cause its dropping in surgery, which decreases efficiency and increases infection as well.

In medical applications, the function of a screw is to fix a bone plate 4 or a web plate in various shapes and let a bone 5 heal. The sizes of screws have limitations due to the required bone reparation or other operations. Generally speaking, the outer diameter “c” of the shank 13 ranges from 1 mm to 5 mm, i.e. 1.5 mm, 2 mm, 2.4 mm; the diameter “a” of the head is 2.4 mm, 3 mm.

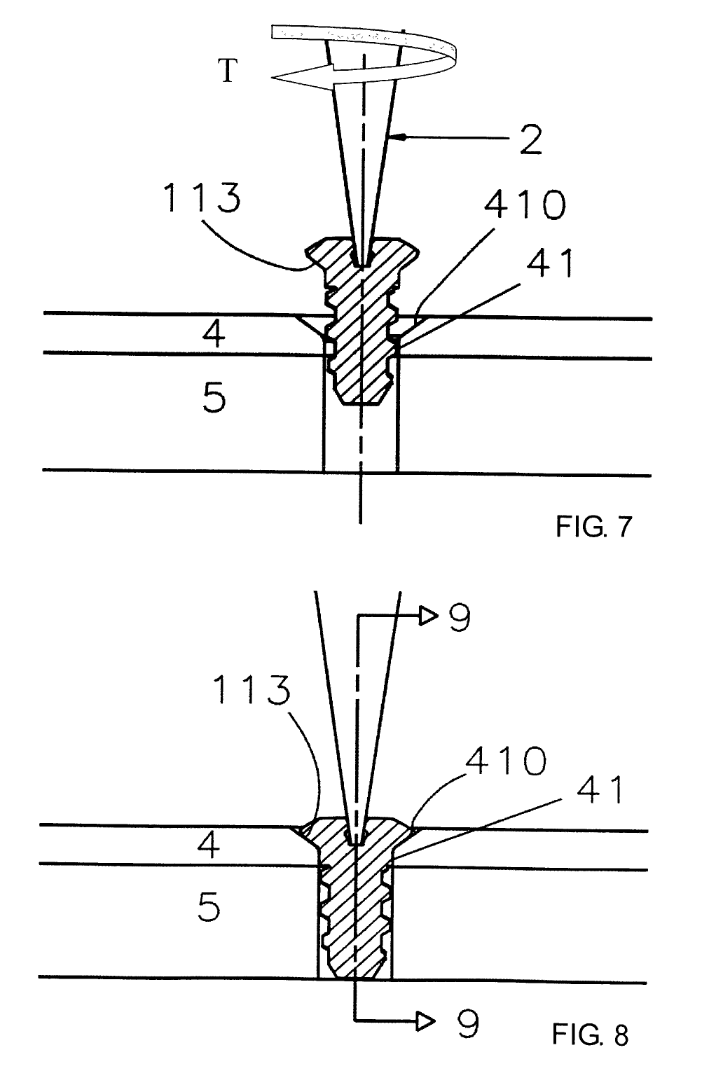

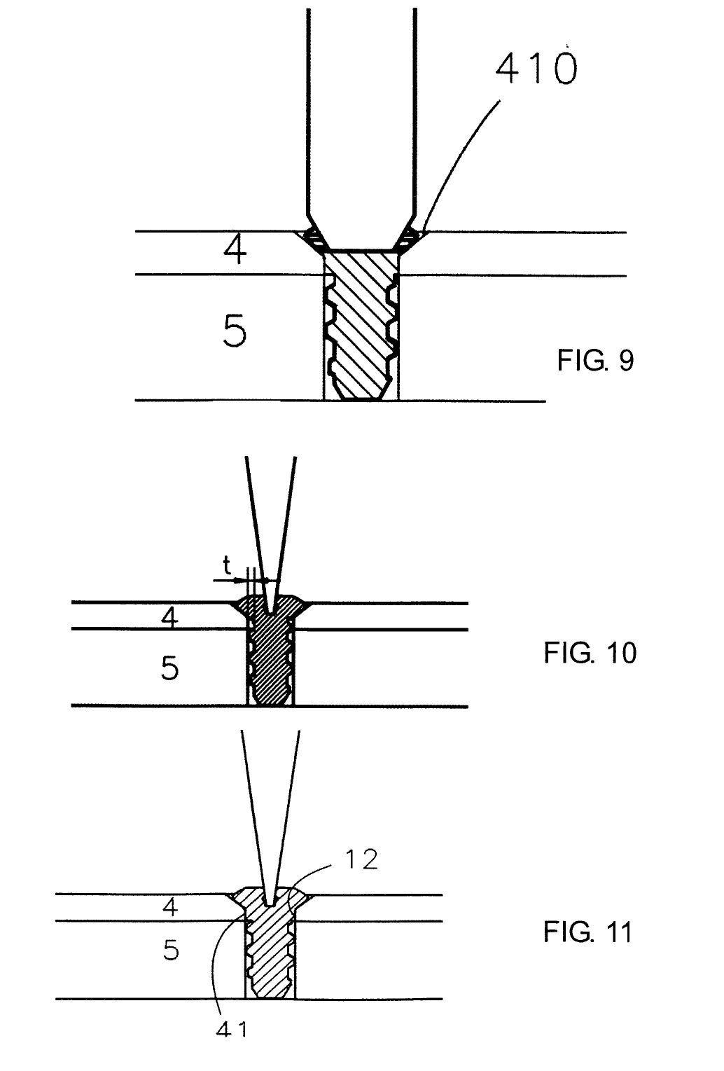

FIG. 7 shows a minus-type screw in the process of being locked into a bone plate and bone, wherein the bone plate 4 has several flat head screws 41 and formed with pilot holes 410. The slant of the tapered lower ring 113 of the head 11 is designed to be the same as that of the pilot hole 410. In FIGS. 8 and 9, it is illustrated the first embodiment of the minus-type screw being completely locked into a bone plate or bone. When a torque T is exercised by the screwdriver 2 subjecting the screw 1 to pass the bone plate 4 and to lock into bone tissue 5, the slant of the tapered lower ring 113 joins the pilot hole 410 of the flat head screw 41. By slightly rocking the screwdriver 2 backward and forward, it can be removed from the slot 3 of screw 1.

Referring to FIG. 4, the implementation is different from the traditional design where diameter “b” of the neck 12 is the same as the inner diameter “d” of shank 13, but the outer diameter “c” of the shank 13 with the aim to reinforce the head and to avoid failure of the head 11 or even of the thread per se resulted from improper stress. In case of such situations, the surgical time will be longer, and the risk of infection will be greater. Therefore, in this invention, the diameter “b” of the head 12 is increased to be same as that of the outside diameter “c” of the shank 13 to minimize the head 11 from risks of being damaged.

Referring to FIGS. 4 and 10, in the past, the diameter “b” of the head is the same as that of the inner diameter “d” of the shank in design, such that when locking the bone plate 4 onto the bone 5, all the torque is concentrated on the thread and there is a clearance t between the neck 12 and the flat head 41, resulting in a rather poor friction effect; more screws are used in the past practices to prevent from loosening. Referring to FIGS. 4 and 11, in this invention, the inventor enlarges the diameter “b” to match with diameter “c”, leaving no clearance between the screw neck 12 and the diameter of the flat head 41 for enhancing friction effect and increasing the coupling effect between the threads and bone threads.

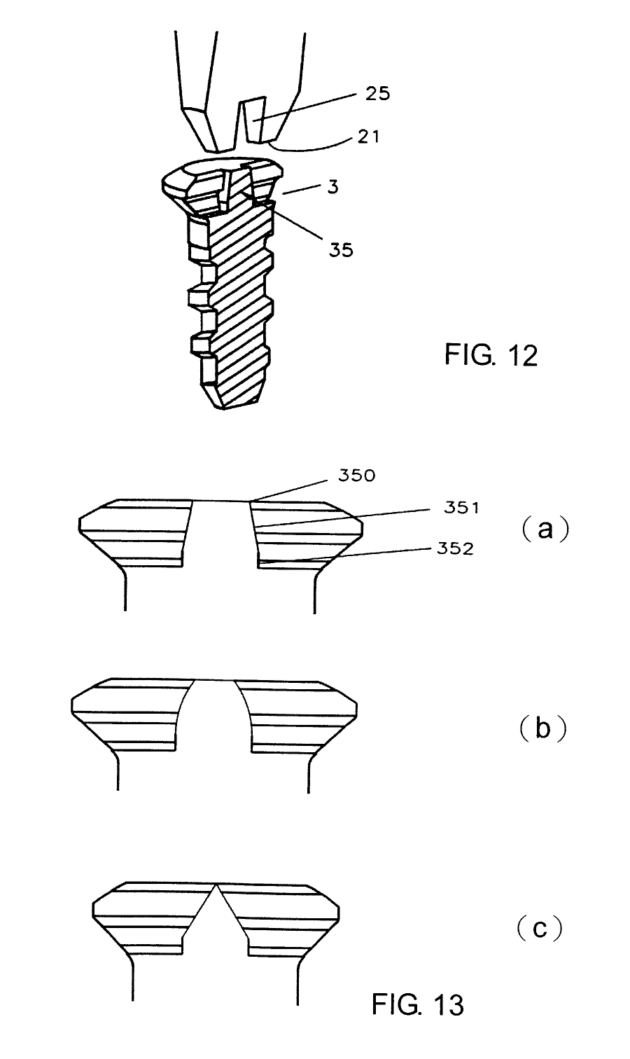

FIG. 12 shows the second embodiment of the present invention, which is an implementation with positioning ribs added to the minus-type screw and the Philips-type screw. In the slot 3 of the head 11, a positioning rib 35 is intentionally added to achieve the object of positioning the screwdriver 2 and aligning with the center. Similarly, a cut 25 is made at the center of the tip 21 to match with it for proper positioning and better tightness.

As shown in FIGS. 13(a), (b), (c), the depth and width equals the positioning rib 35 at the center of slot 3, a narrow tip 350 is set in place for easy insertion into the slot 25 of the tip 21. Beneath the tip 350 is the rib 351 extending in a linear curve or a nonlinear curve to reach the wide bottom tip 352 for final positioning of the screw 2. Regardless of the types of curve of the positioning rib 35, the common feature is that the lower tip 352 is vertical allowing proper positioning and tight engagement to the slot 25 of screw 2.

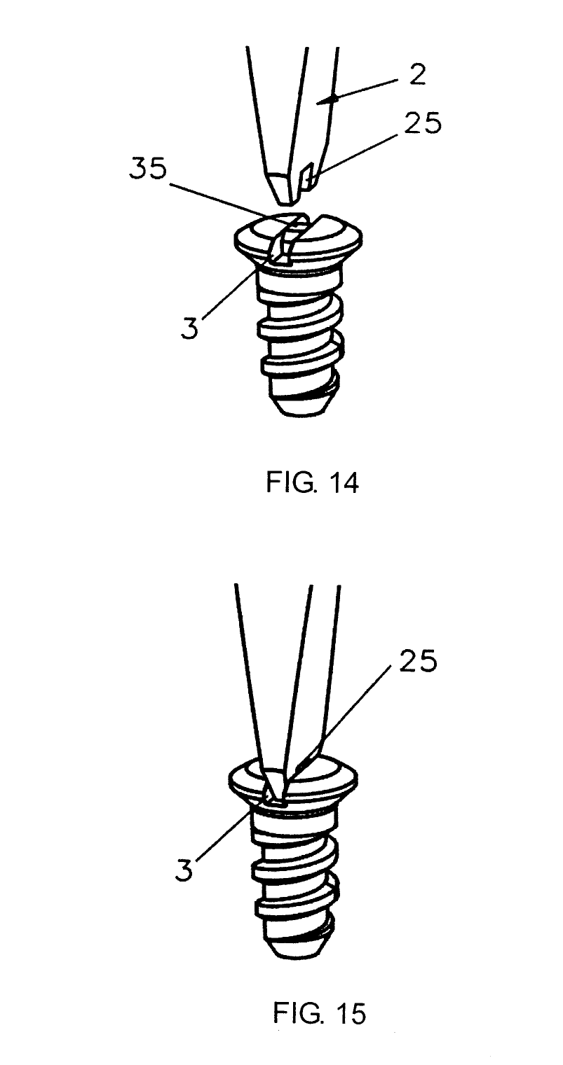

Further to the design in the second embodiment in enhancing the positioning and engagement between the positioning rib 35 and the screw 2, as shown in FIGS. 14 15 of the third embodiment, the holding section 31, middle section 32 and fixing section 33 are omitted in the design, i.e., the positioning rib 35 is placed in the slot 3 of the head 11 (without the above-mentioned holding section, middle section and fixing section). The positioning and engagement effect is achieved by the cut 25 of the screw 2 for matching with the positioning rib 35.

The screw material used in designing the present invention is non-magnetic; presently polymers and/or copolymers made from alpha-hydroxy acid are used. The key point is that, in case of a different material is to be used, whether it is bio-absorbable or not, it should be biocompatible. Plastic, wood, resin and some non-magnetic metals such as titanium, copper and stainless steel are recommended.

The following ratios are recommended for design of the screws of the present invention:

1 the ratio of outer diameter “c” of the shank 13 to diameter “a” of the head 11 should be less than or equal to 0.9;

2 the ratio of thickness e of the head 11 to diameter “a” should be 0.2˜0.4;

3 the ratio of thickness e of the head to outer diameter “c” of the shank should be 0.2˜0.5;

4 the ratio of thickness f from the tip 110 to the center of the middle ring 112 to thickness e of the head should be 0.2˜0.4 (Note: a bulge is formed after healing of the wounds; from the viewpoint of aesthetics, the lower the ratio is, the less apparent the bulge is).

In categorizing the products carrying the present invention, screws of different sizes are stored in boxes of different colors. For example shank diameter being 2 mm is in the yellow box; shank diameter being 2.4 is in the red box. Similarly, colors of screwdrivers matching that of screws may be implemented to minimize the risk of mistakes.

The above statements and drawings are only meant for detailed presentation of the embodiments of the present invention and should not constitute a limitation in the implementation of the present invention; any device with equivalent varieties and modifications within the scope of the present patent application shall fall in the scope of the present invention.

References

Cohen J, Wulff J (1972): Clinical failure caused by corrosion of a Vitallium plate J Bone Jt Surg 54-A: 617-628

Cochran G V B (1969): effect of internal fixation plates on mechanical deformation of bone Surg Forum 20: 469-471

Higgins N A (1954): Condensation polymers of hydroxyacetic acid U.S. Pat. No. 2,676,945

Kawahara H, Hirabayashi H, Shikita T (1980): Single crystal alumina for dental implants and bone screws J Biomed Master Res 14: 597-605

Leenslag J W, Penning A J, Bos R R M, Rozema F R, Boering G (1987): Resorbable materials of poly (L-lactide) VII. In vitro degradation Biomaterials 8:311-314

Muller E, Allgower M, Schneider R, Willlenegger H (1979): Manual of Internal Fixation, Springer-Verlag, Berlin

Schaztker J, Tile M (1987): The Rationale of Operative Fracture Care, Springer-Verlag, Berlin

Slatis P, Karaharju E, Holmstrom T, Ahonen J, Paavolainen P (1978): Structure changes in intact bone after application of rigid plates with and without compression J Bone Jt Surg 60-A: 516-522

Tonino A J, Davidson C L, Klopper P J, Linclau L A (1976): Protection from stress in bone and plastic plates in dog J Bone Jt Surg 58-B: 107-113

Uhthoff H K, Dubuc F L (1971): Bone structure changes in the dog under rigid internal fixation Clin Orthop 81: 165-170

What is claimed is:

1. A device for fixing bones, comprising a biocompatible screw and a complementary screwdriver, wherein the screw comprises a head formed with at least one slot, a screw neck, and a shank; the slot comprises of a holding section, a middle section and a fixing section from top to bottom.

2. The bone-fixing device of the claim 1, wherein the head and the shank is separated by the neck; the head includes a top flat, conical upper ring, middle ring and tapered lower ring; the neck of the screw is substantially a cylindrical body, which extends from the tapered lower ring to the shank of the screw.

3. The bone-fixing device of the claim 1, wherein the screw is a minus-type screw when one straight slot is formed on the head, or a Philips-type screw when two normal and symmetrical slots are formed on the head.

4. The bone-fixing device of the claim 1, wherein the screw is made of non-magnetic material.

5. The bone-fixing device of the claim 1, wherein the screw is made of bio-absorbable material.

6. The bone-fixing device of the claim 2, wherein the screw diameter ranges from 1 mm to 5 mm.

7. A bone-fixing device, comprising: a biocompatible screw and a complementary screwdriver, wherein the screw comprises a head formed with at least one slot, a neck, and a shank, characterized in that, the diameter of the neck equals the outer diameter of the shank.

8. A bone-fixing device, comprising: a biocompatible screw and a complementary screwdriver, wherein the screw comprises a head formed with at least one slot, a neck, and a shank, characterized in that, a positioning rib is provided in the slot.

9. The bone-fixing device of the claim 8, wherein the positioning rib includes a narrow tip and a linear or non-linear rib body extending from the top to the wider lower part.

10. The bone-fixing device of the claim 9, wherein the positioning rib has a vertical lower part.

11. A bone-fixing device, comprising a biocompatible screw and a complementary screwdriver, wherein the screwdriver has a slant tip, a shank, and a handle, which tip being provided to be inserted into the slot.

12. The bone-fixing device of the claim 11, wherein the screw tip is to match the number of slots formed on the head to be a minus- or Philips-type screw.

13. The bone-fixing device of the claim 8 or 12, wherein the screw tip has a cut at a location corresponding to the positioning rib.
